(19) United States
(12) Patent Application Publication
(10) Pub. No.: US 2018/0054599 A1
(43) Pub. Date: Feb. 22, 2018

(54) HUMAN GROWTH TRACKING APPARATUS, SYSTEM AND METHOD

(71) Applicant: SCS COMPANIES, LLC, Wellsville, UT (US)

(72) Inventor: Chelsie Sanders, Wellsville, UT (US)

(21) Appl. No.: 15/243,705

(22) Filed: Aug. 22, 2016

(51) Int. Cl. H04N 7/18; A41B 13/06; A61B 5/00; G09F 7/18; G09F 23/00

(52) U.S. Cl. CPC H04N 7/183; A41B 13/06; G09F 23/00; G09F 7/18; A61B 5/0077

(57) ABSTRACT

An apparatus, system and method for tracking growth of a human over time and in correlation with various notable events, for example, the size of an infant when the infant turns one month old, the size of an infant when the infant first rolls over, the size of an infant when the infant experiences a holiday for the first time, or the size of an expectant mother's expanded torso when she is 36 weeks into her pregnancy, through image capture of the growing human located proximate correlative indicia placed upon a display face of a flexible tracking sheet and during or close-in-time to the occurrence of a notable event.

### BACKGROUND

The journey from infancy to full-grown adulthood is, for many, often filled will notable events or milestones that occur during the growth period. Moreover, pregnancy, in a somewhat similar manner, is also often marked by prominent occurrences as a woman's torso grows to full-term development. The human characteristics for physical growth has long prompted people to strive to capture or otherwise document growth progression. However, growth tracking and milestone capturing ordinarily occur separately and distinctly. Hence there is a need for an efficient and effective apparatus, system and method for tracking human growth while simultaneously documenting notable occurrences in human development.

A described aspect provides a human growth tracking apparatus comprising: a flexible tracking sheet, the flexible tracking sheet having at least one display face, wherein the at least one display face covers an area of at least four square feet, the at least four square foot area being defined by at least two perpendicular dimensions, each of the at least two perpendicular dimensions being at least two feet in length; and a plurality of indicia located on the at least one display face of the flexible tracking sheet, wherein the plurality of indicia corresponds to notable events that occur during a period of growth of a human.

Another described aspect provides a system for tracking human growth in correlation with notable events, the system comprising: an imaging device, the imaging device configured to capture at least one image of a growing human, wherein the imaging device includes a storage medium configured to store the at least one captured image of the growing human, and further wherein the at least one image is captured during or close-in-time to the occurrence of a notable event during a period of growth of the human; and a human growth tracking apparatus, the human growth tracking apparatus including: a flexible tracking sheet, the flexible tracking sheet having at least one display face, wherein the at least one display face covers an area of at least four square feet, the at least four square foot area being defined by at least two perpendicular dimensions, each of the at least two perpendicular dimensions being at least two feet in length; and a plurality of indicia located on the at least one display face of the flexible tracking sheet, wherein the plurality of indicia corresponds to notable events that occur during the period of growth of the human.

Yet another described aspect provides a method of tracking human growth, the method comprising: providing a human growth tracking apparatus, the human growth tracking apparatus including: a flexible tracking sheet, the flexible tracking sheet having at least one display face, wherein the at least one display face covers an area of at least four square feet, the at least four square foot area being defined by at least two perpendicular dimensions, each of the at least two perpendicular dimensions being at least two feet in length; and a plurality of indicia located on the at least one display face of the flexible tracking sheet, wherein the plurality of indicia corresponds to notable events that occur during a period of growth of a human; locating a human proximate the plurality of indicia located on the at least one display face of the flexible tracking sheet during or close-in-time to the occurrence of a notable event during the period of growth of the human; signifying specific indicia that correlatively identifies the particular notable event being identified as correspondingly relevant for tracking growth of the human; providing an imaging device; capturing at least one image of the growing human, as the growing human is located proximate the plurality of indicia located on the at least one display face of the flexible tracking sheet, wherein the image is captured during or close-in-time to the occurrence of the notable event during the period of growth of the human; and storing, at least temporarily, the at least one captured image of the growing human, wherein the captured image is stored during or close-in-time to the occurrence of the notable event during the period of growth of the human.

### BRIEF DESCRIPTION OF THE DRAWINGS

The described aspects are best understood from the following detailed description when read in connection with the accompanying drawing(s). Included in the drawing(s) are the following figure(s):

FIG. 1 is a top view of an embodiment of a human growth tracking apparatus;

FIG. 2 is a top perspective view of the embodiment of a human growth tracking apparatus depicted in FIG. 1;

FIG. 3 is a top view of the embodiment of a human growth tracking apparatus depicted in FIG. 1 being used in a manner to track the growth of a human;

FIG. 4 is a top view of another embodiment of a human growth tracking apparatus;

FIG. 5 is a top view of the embodiment of a human growth tracking apparatus depicted in FIG. 4 being used in a manner to track the growth of a human;

FIG. 6 is a top view of still another embodiment of a human growth tracking apparatus;

FIG. 7 is a top view of the embodiment of a human growth tracking apparatus depicted in FIG. 6 being used in a manner to track the growth of a human;

FIG. 8 is a top view of yet an embodiment of a human growth tracking apparatus;

FIG. 9 is a top view of the embodiment of a human growth tracking apparatus depicted in FIG. 8 being used in a manner to track the growth of a human;

FIG. 10 is a top view of a further embodiment of a human growth tracking apparatus;

FIG. 11 is a top view of the embodiment of a human growth tracking apparatus depicted in FIG. 10 being used in a manner to track the growth of a human;

FIG. 12 is a top view of still yet a further embodiment of a human growth tracking apparatus;

FIG. 13 is a top view of the embodiment of a human growth tracking apparatus depicted in FIG. 12 being used in a manner to track the growth of a human;

FIG. 14 is a top view of the embodiment of a human growth tracking apparatus depicted in FIG. 12 being used in another anner to track the growth of a human;

FIG. 15 is a top view of the embodiment of a human growth tracking apparatus depicted in FIG. 12 being used in still another manner to track the growth of a human; and

FIG. 16 is a top view of an embodiment of a system for tracking human growth.

### DETAILED DESCRIPTION

While this disclosure contains many specific details, it should be understood that various changes and modifications may be made without departing from the scope of the technology herein described and the appended claim(s). The scope of the technology shall in no way be construed as being limited to the number of constituting components, the corresponding relationship, if any, of constituting components, the materials thereof, the shapes thereof, the relative arrangement thereof, the temperature employed, the order of combination of constituents thereof, etc., and are disclosed simply as examples. The depictions and schemes shown herein are intended for illustrative purposes and shall in no way be construed as being limiting in the number of constituting components, connectivity, methodological steps, the materials thereof, the shapes thereof, the relative arrangement thereof, the order of system interactions thereof, etc., and are disclosed simply as an aid for understanding. The features and advantages of the described embodiments are illustrated in detail in the accompanying drawing(s), wherein like reference numerals refer to like elements throughout the drawing(s). As a preface to the detailed description, it should be noted that, as used in this specification and the appended claim(s), the singular forms “a”, “an” and “the” include plural referents, unless the context clearly dictates otherwise.

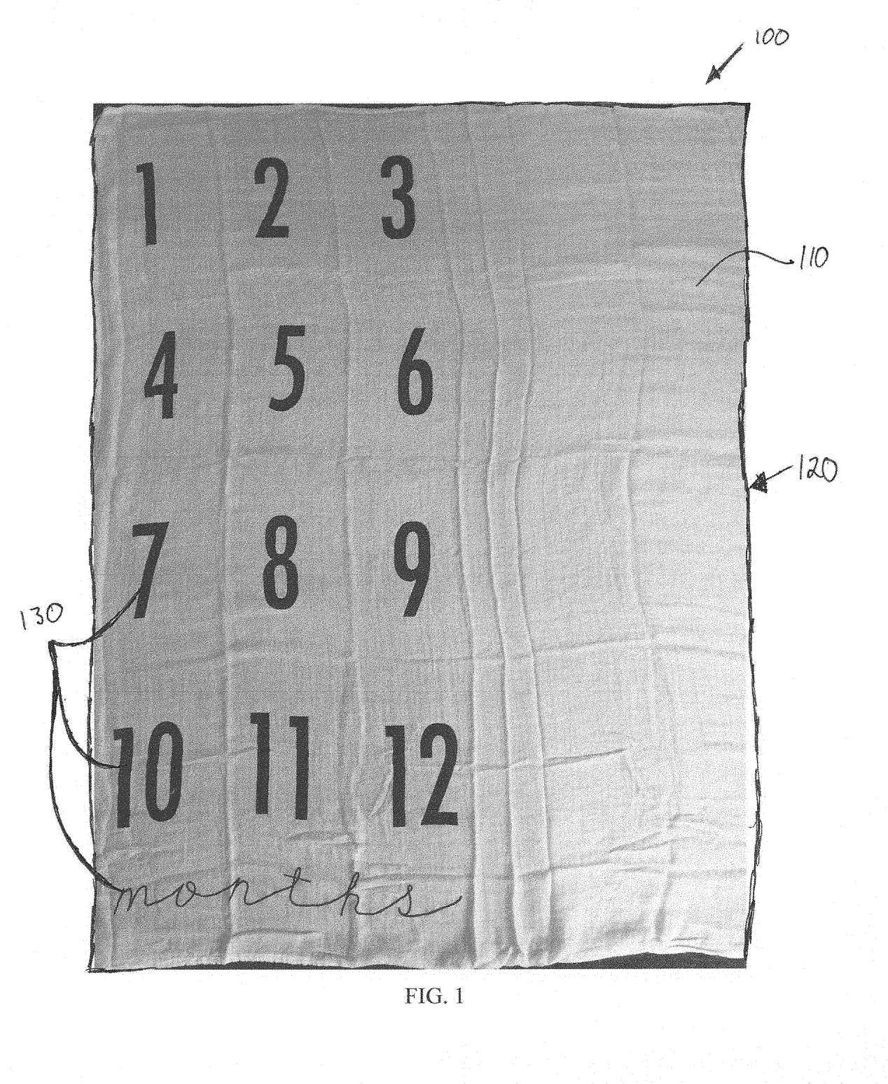

With reference to the drawing(s), FIG. 1 depicts a top view of an embodiment of a human growth tracking apparatus 100. The human growth tracking apparatus 100 may comprise a flexible sheet 110, formed for example of cloth, linen, woven or felted fabric made from wool, cotton, or a similar fiber, or other textile material. In addition, the flexible sheet 110 may be fanned of plastic or other polymeric material or thin flexible metal materials or foils, or any material or combination of materials that will facilitate a lightweight and flexible sheet 110.

The flexible sheet 110 of the human growth tracking apparatus 100 includes at least one display face 120. The term “face,” as used herein, refers, inter alia, to a designated side or portion of the flexible sheet 110 of the human growth tracking apparatus 100 that is configured for depiction of components located thereon. The flexible sheet 110 and/or the display face 120 may comprise a uniform coloring or shading scheme and/or may contain multiple colors or patterns. In addition, the display face 120 of the flexible sheet 110 may be configured to prominently present, show or otherwise exhibit a plurality of indicia 130 located on the at least one display face 120.

The plurality of indicia 130 may be comprised of, formed or otherwise fashioned in any manner that effectuates visual presentation of information corresponding to notable events that may occur during a period of growth of a human. Moreover, the plurality of indicia 130 may be presented in the form of signs, codes, cryptograms, or other communication markers, such as alphanumeric symbols. For example, as exemplarily depicted in FIG. 1 the plurality of indicia includes silk-screened numerals (1-12), as well as the cursive scripted word “months” silk-screened onto the display face 120 of a flexible sheet 110 of bamboo cotton muslin in the form of a swaddle blanket.

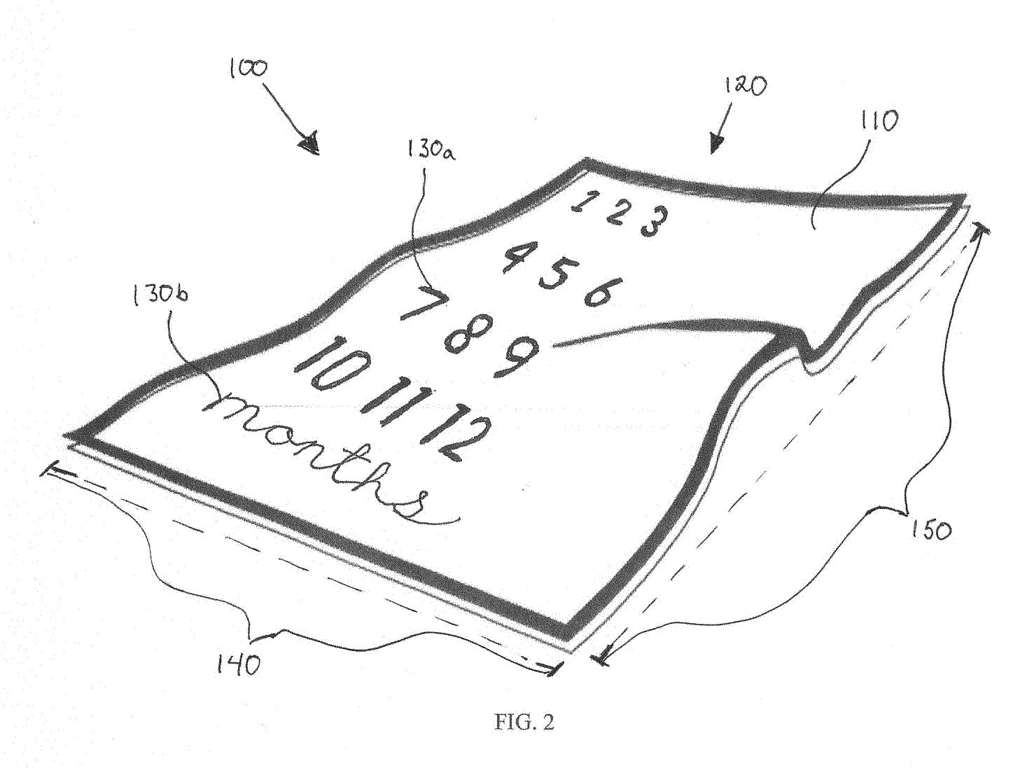

With continued reference to the drawings, FIG. 2 depicts a top perspective view of the embodiment of a human growth tracking apparatus 100 depicted in FIG. 1. The at least one display face 120 of the human growth tracking apparatus 100 covers an area of at least four square feet, the at least four square foot area being defined by at least two perpendicular dimensions (dimension 140 and dimension 150), each of the at least two perpendicular dimensions (140 and 150) being at least two feet in length. Those of ordinary skill in the art will appreciate that the two perpendicular dimensions (140 and 150) may intersect at a center of a circle (such that each dimension is the same length and representative of a diameter of the circle), may intersect at a foci of an ellipse, may form the base and height of an isosceles triangle or may be oriented in any manner such that the display face 120 includes at least four square feet of surface area and each of dimension 140 and dimension 150 are at least two feet in length. A four square foot surface area helps to facilitate better tracking of human growth.

As further shown in FIG. 2, the plurality of indicia 130 corresponds to notable events that occur during a period of growth of a human. For example, each of the printed numbers 1-12 represents a month of the year, with 1 being representative of January, 2 being representative of February, and so on, so that each number is an indicia 130a. The indicia 130b identifies, through wording, that this human growth tracking apparatus embodiment 100 tracks “months.”

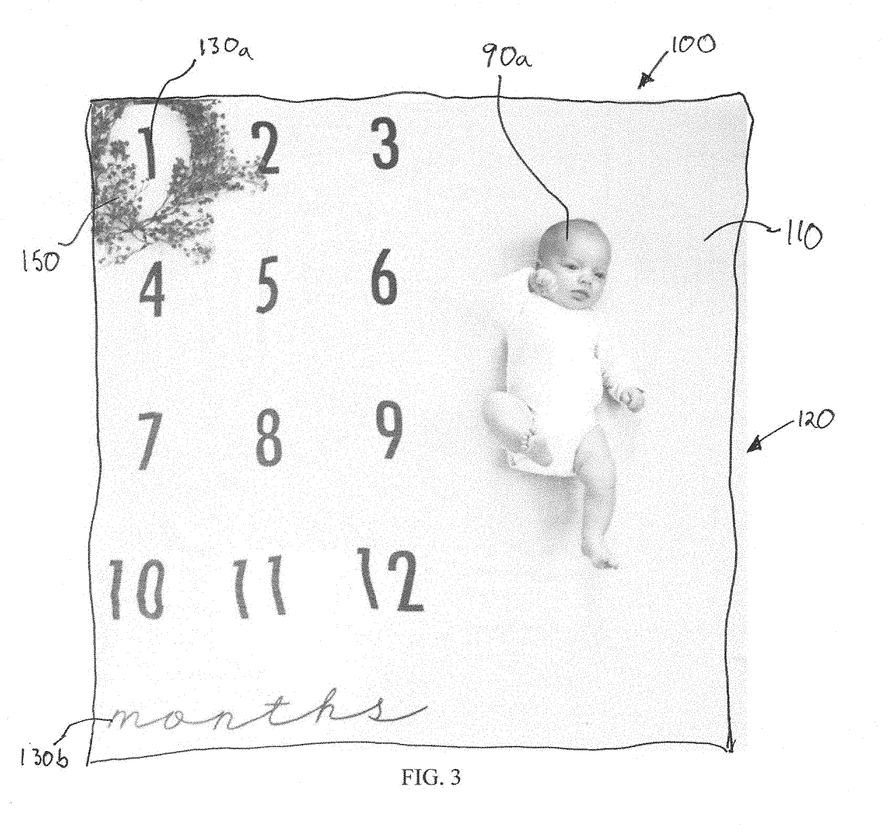

With additional reference to the drawings, FIG. 3 depicts a top view of the embodiment of a human growth tracking apparatus 100 depicted in FIG. 1 being used in a manner to track the growth of a human, in this case, an infant baby 90a. Once the human growth tracking apparatus 100 is provided, the growth-size of a human can be tracked in correlation with the occurrence of a notable event. Accordingly, the human infant 90a may be located or otherwise positioned, oriented, placed and/or set-upon the display face 120 of the flexible sheet 110. The location of the human infant proximate the display face 120 of the flexible sheet 110 may be during or close-in-time to the occurrence of the notable event. For example, if the size of the human infant 90a is desired to be tracked in conjunction with the human infant's one-month birthday then the human infant 90a may be located proximate to the display face 120 of the flexible sheet 110 either on the date of the human infant's one-month birthday, or within a day or so prior to or following that date, so that the size of the human infant will correspond accurately with growth up to that one-month birthday date. In addition to proximately locating the human infant 90a next to or on the human growth tracking apparatus embodiment 100, a user may also signify, circle, point-to, highlight, emphasize, or otherwise recognize specific indicia 150 that correlatively identifies the particular notable event being identified as being correspondingly relevant for growth tracking. Thus, in continuance of the example, a user may signify the first month (the silk-screen printed numbe “1” indicia 150 on the display face 120 of the human growth tracking apparatus embodiment 100), to correspondingly identify that the human infant 90a is approximately one-month old, or otherwise celebrating a one-month birthday at the time of the occurrence of growth tracking. An imaging device, such as a camera or other device capable of capturing an image, may be provided to capture at least one image of the human infant 90a located proximate the signified indicia 150, of the plurality of indicia, thereby identifying the human infant 90a as being approximately one month old in the captured image. For purposes of documenting or otherwise tracking the size of the human infant 90a as captured in correlation with the notable moment, the captured image may be at least temporarily stored, such as on film or on a digital hard drive or flash drive, during or close-in-time to the occurrence of the notable event during the period of growth of the human infant 90a.

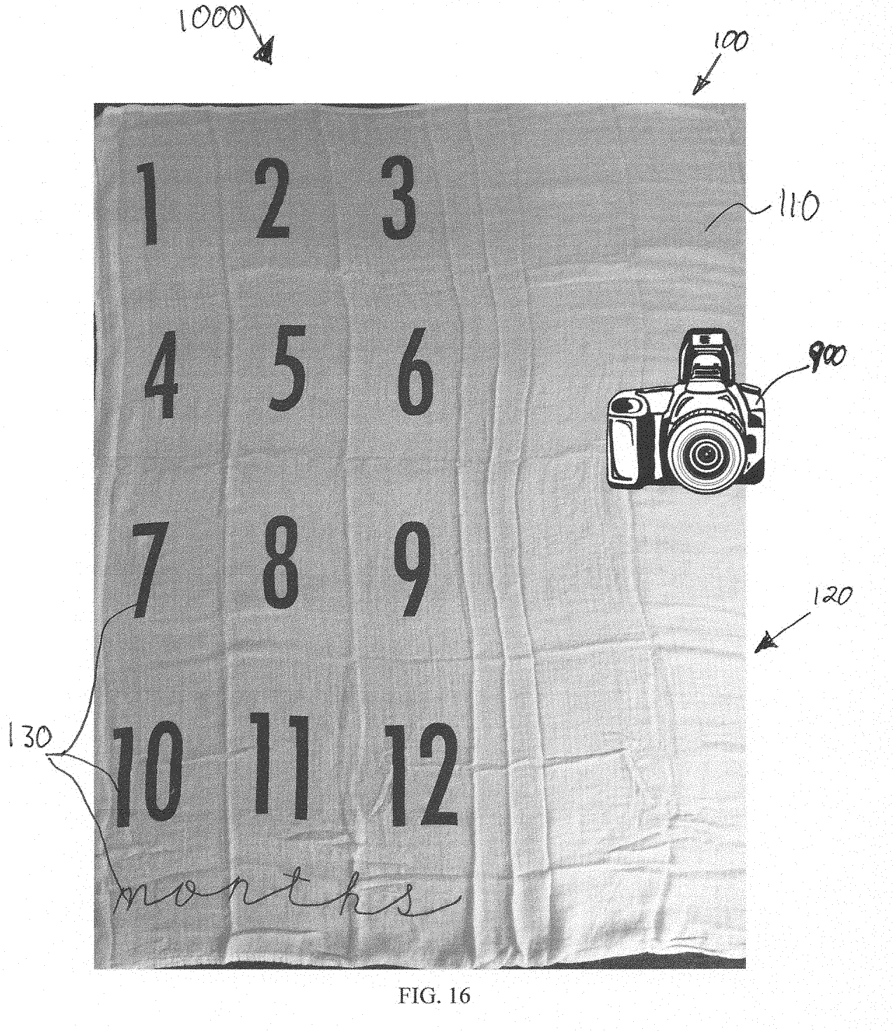

With still continued reference to the drawings, FIG. 16 depicts a top view of an embodiment of a system 1000 for tracking human growth. The system particularly comprises an imaging device 900, such as a camera or any other device capable of capturing an image. The imaging device 900 may be configured to capture at least one image of a growing human, such as a human infant 90a of FIG. 3, wherein the imaging device 900 includes a storage medium configured to store the at least one captured image of the growing human. Examples of a storage medium comprise film, magnetic tape, hard drives, floppy disks, compact disks, digital video disks, flash drives or any other medium capable of storing a captured image. The system 100 for tracking human growth further includes a human growth tracking apparatus, such as the human growth tracking apparatus 100 described and depicted in regard to FIGS. 1-3, or any other operable embodiment as set forth herein or equivalently understood by those of ordinary skill in the art.

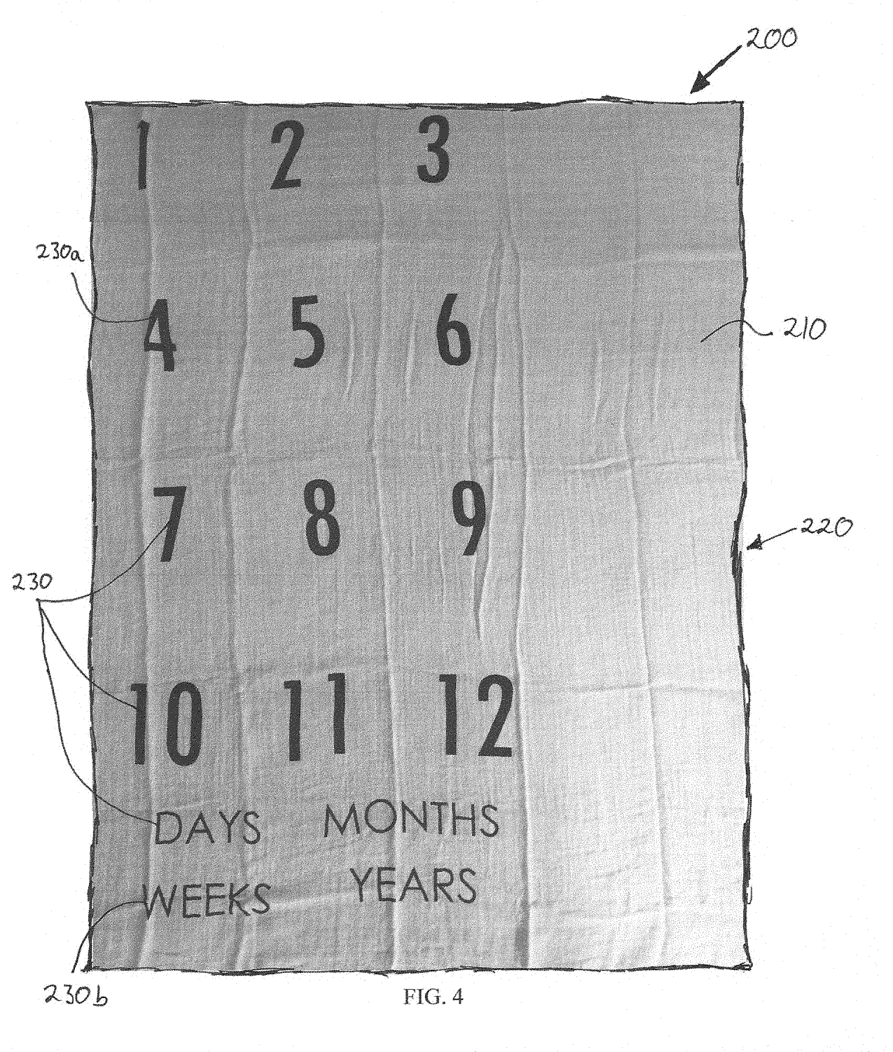

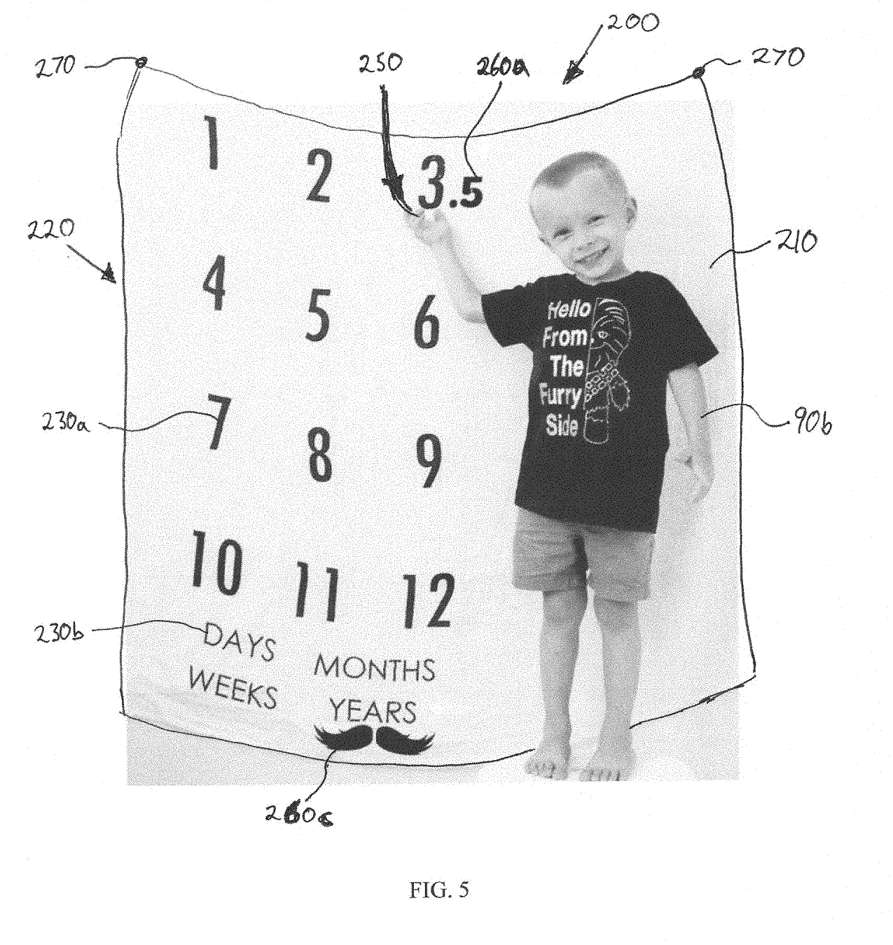

Additional embodiments of a human growth tracking apparatus 200, 300, 400, 500 and 600 will now be discussed in relations to drawing FIGS. 4-15, along with corresponding methodology and systems utilizing such embodiments. In particular, FIG. 4 depicts a top view of another embodiment of a human growth tracking apparatus 200. This embodiment 200 is similar to embodiment 100, but also adds additional indicia pertaining to “Days,” “Weeks” and “Years,” thereby affording a potential user the opportunity to track human growth in correlation to additional varying notable occurrences. For instance, FIG. 5 depicts a top view of the embodiment of a human growth tracking apparatus 200 depicted in FIG. 4 being used in a manner to track the growth of a human child 90b. Also depicted is the proximate location of the human growth tracking apparatus 200 near a standing human child 90b. In this instance, the human growth tracking apparatus 200 may be hung or otherwise suspended vertically support components 270. Furthermore, as depicted the human child 90 may be the means for signifying 250 which particularly indicia is/are correlatively appropriate for the image capturing the growth size of the human child 90b. Moreover, as exemplarily depicted, use of extraneous indicia, such as the mustache symbol 260c under the “years” indicia, and/or the “0.5” half year symbol after the third year indicia, may serve to further clarify the time occurrence being captured in relation to the size of the human child 90b at the time of the image capture. Other component elements, such as the display face 220 and the flexible sheet 210 are similar to comparable elements of the human growth tracking apparatus embodiment 100 described and depicted in regard to FIGS. 1-3.

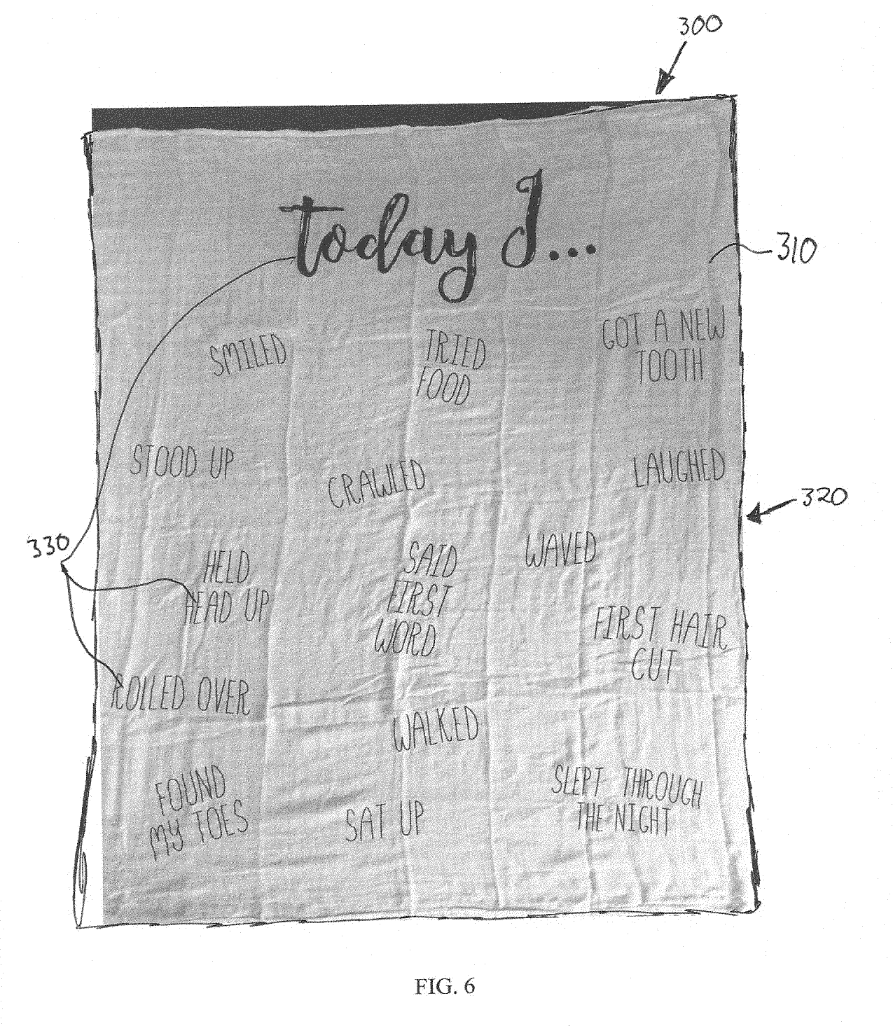

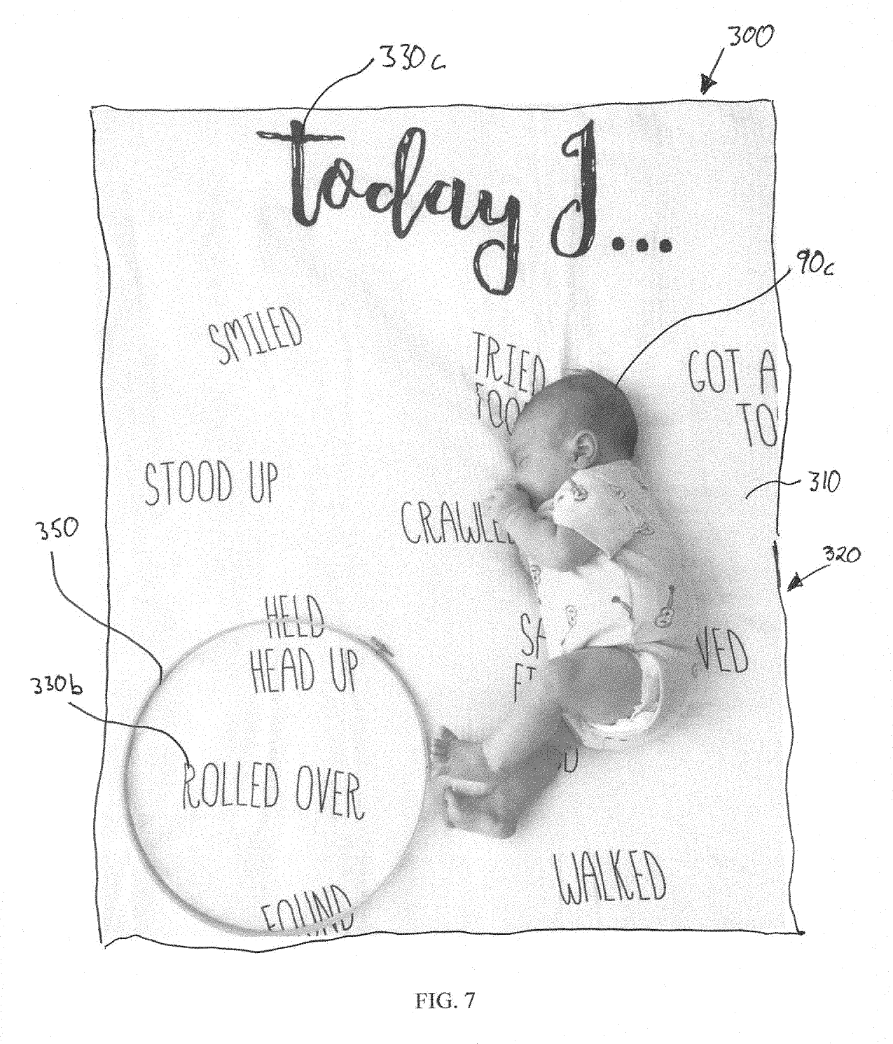

Referring further to the drawings, FIGS. 6 and 7 depict a top view of an embodiment of a human growth tracking apparatus 300 being used in a manner to track the growth of a human infant 90c. Notably, this embodiment is completely independent of time, and instead provides a plurality of indicia 330 focusing instead on milestone happenings, such as when a human infant 90c first crawls. As depicted, a signifying 350 physical wooden hoop device is located around the “Rolled Over” indicia to indicate that, on or about the time the image was captured, the human infant 90c first rolled over and had a body size of that shown in the captured and stored image. IN addition, indicia 330c indicates vastly different font size and type as compared with indicia 330b. The display face 320 and flexible sheet 310 are similar to that described with respect to comparable elements associated with human growth tracking apparatus embodiment 100 described and depicted in regard to FIGS. 1-3.

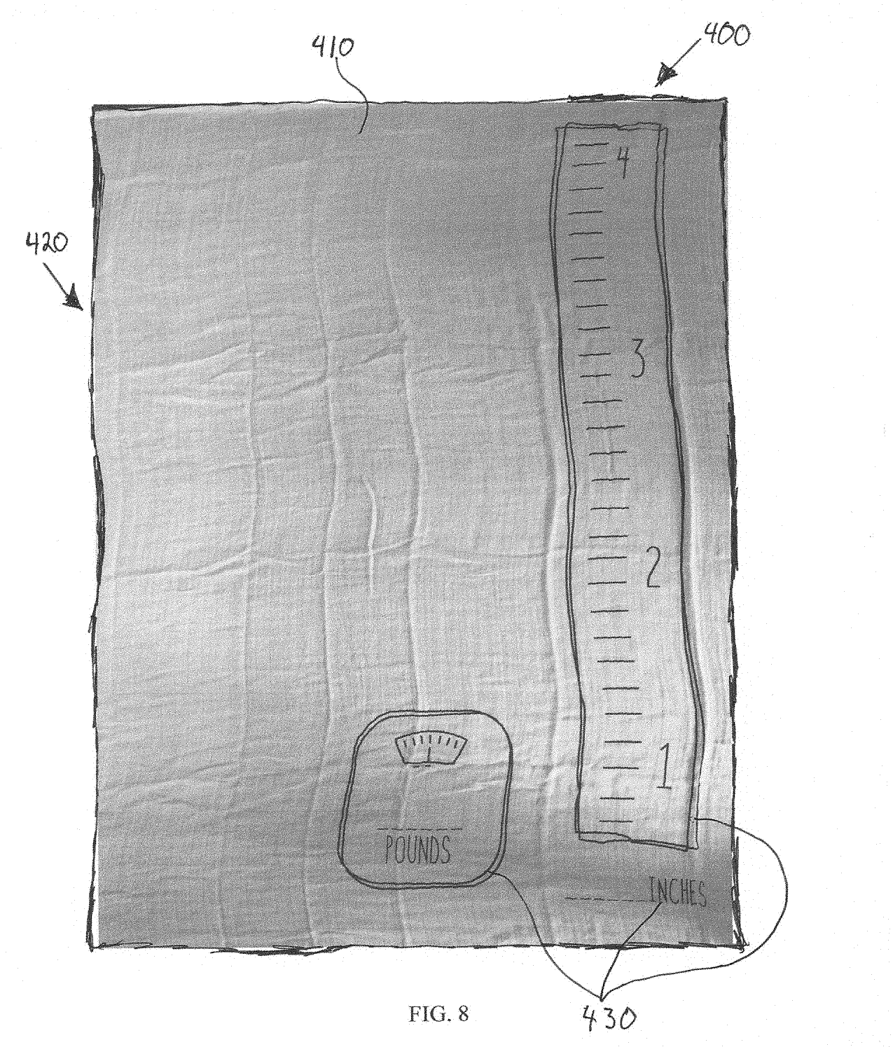

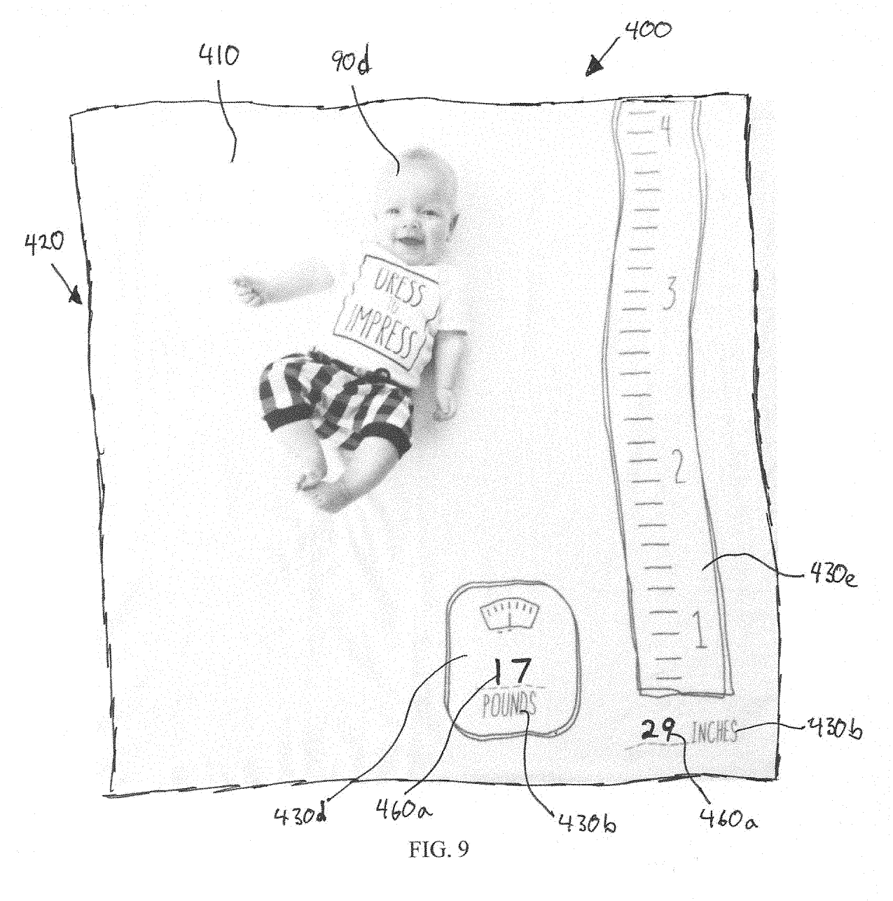

Again, referring further to the drawings, FIGS. 8 and 9 depict a top view of an embodiment of a human growth tracking apparatus 400 being used in a manner to track the growth of a human infant 90d. Notably, this embodiment is completely devoid of time, and instead provides a plurality of, inter alia, pictorial indicia 430 focusing instead on milestone happenings, such as the height and weight of a human infant 90d. Here, as exemplified, a blank spot indicia 460a is purposefully left open on the scale image indicia 430d and under the ruler image indicia 430e and when filled in with informative data indicate that, on or about the time the image was captured, the human infant 90d was the height and weight shown in the captured and stored image. The display face 420 and flexible sheet 410 and indicia 430b are similar to that described with respect to comparable elements associated with human growth tracking apparatus embodiment 100 described and depicted in regard to FIGS. 1-3.

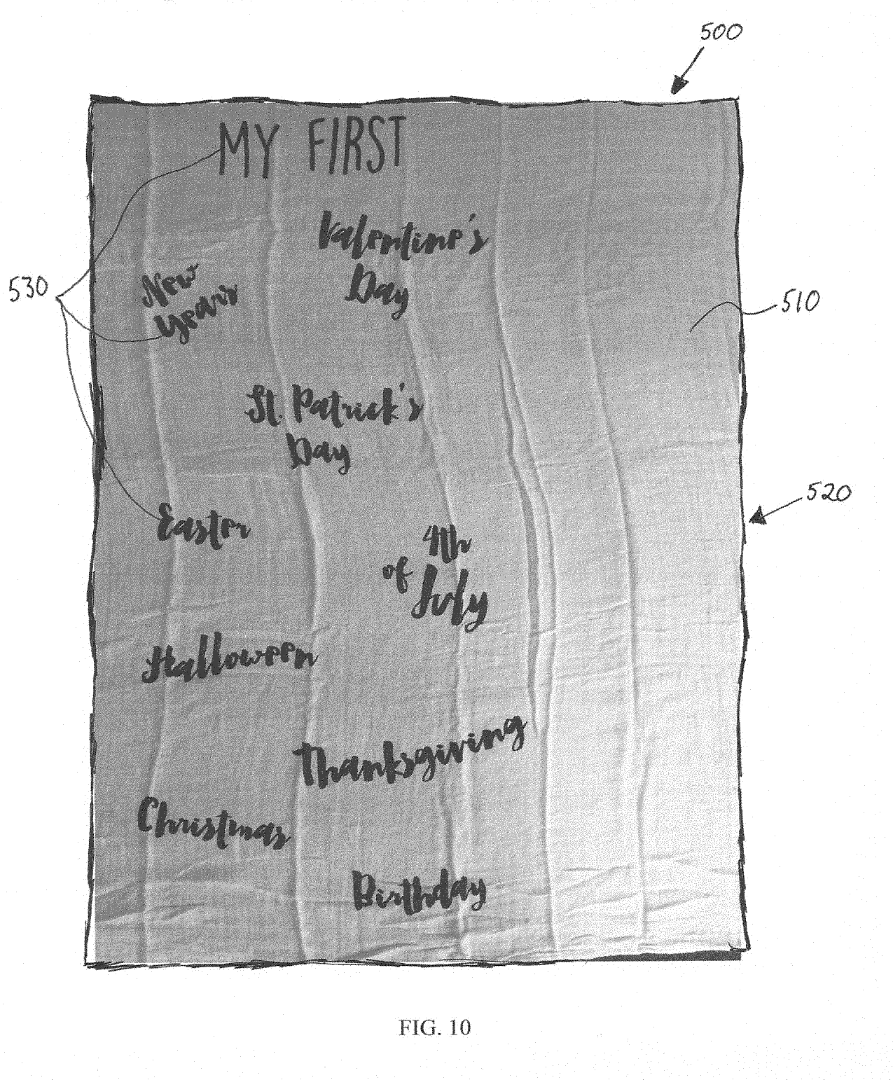

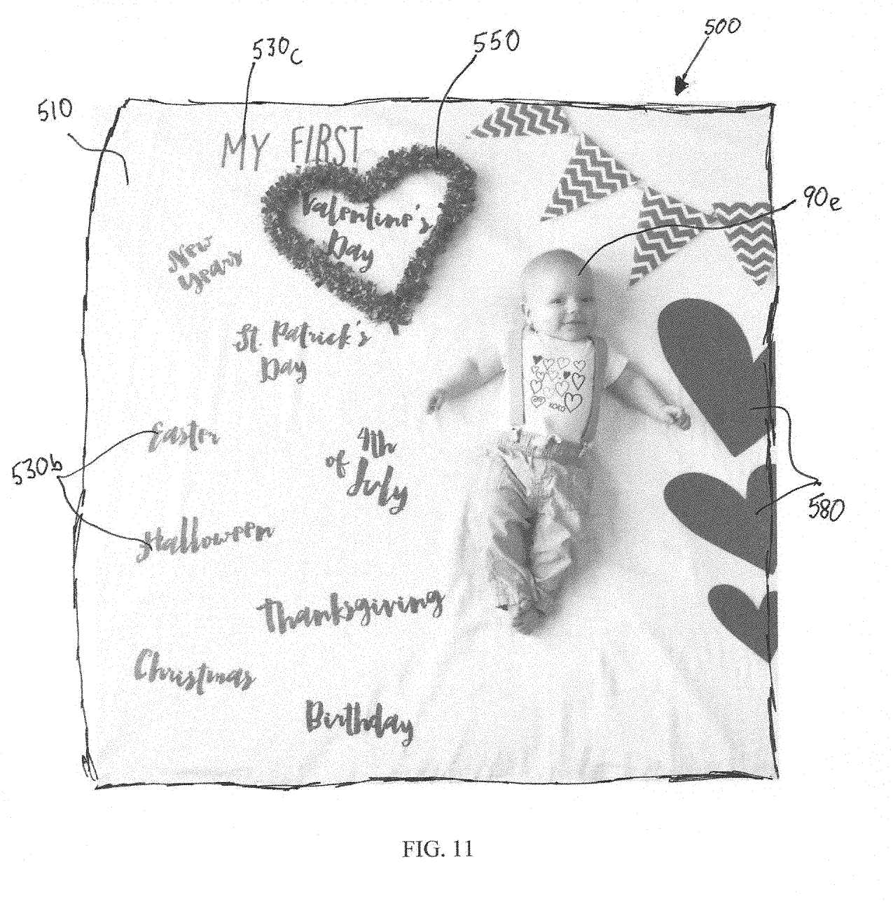

With further reference to FIGS. 10 and 11, a top view of an embodiment of a human growth tracking apparatus 500 is depicted and shown being used in a manner to track the growth of a human infant 90e. This embodiment human growth tracking apparatus 500 focuses all notable events on first experiencing well-known holidays, as set-forth by the “My First” indicia 530c. For example, when a human infant 90e experiences Valentine's Day for the first time, the human infant 90e can be located proximate the holiday indicia 530b and a garland heart 550 can be placed particularly around the “Valentine's Day” indicia signifying that the growth size of the human infant 90e is what it is during or close-in-time to that particular holiday when the image of the same is captured. Moreover, further holiday accoutrements and decorations 580, such as hearts, can also emphasize the holiday milestone. As before, the display face 520 and flexible sheet 510 and indicia 530b are similar to that described with respect to comparable elements associated with human growth tracking apparatus embodiment 100 described and depicted in regard to FIGS. 1-3.

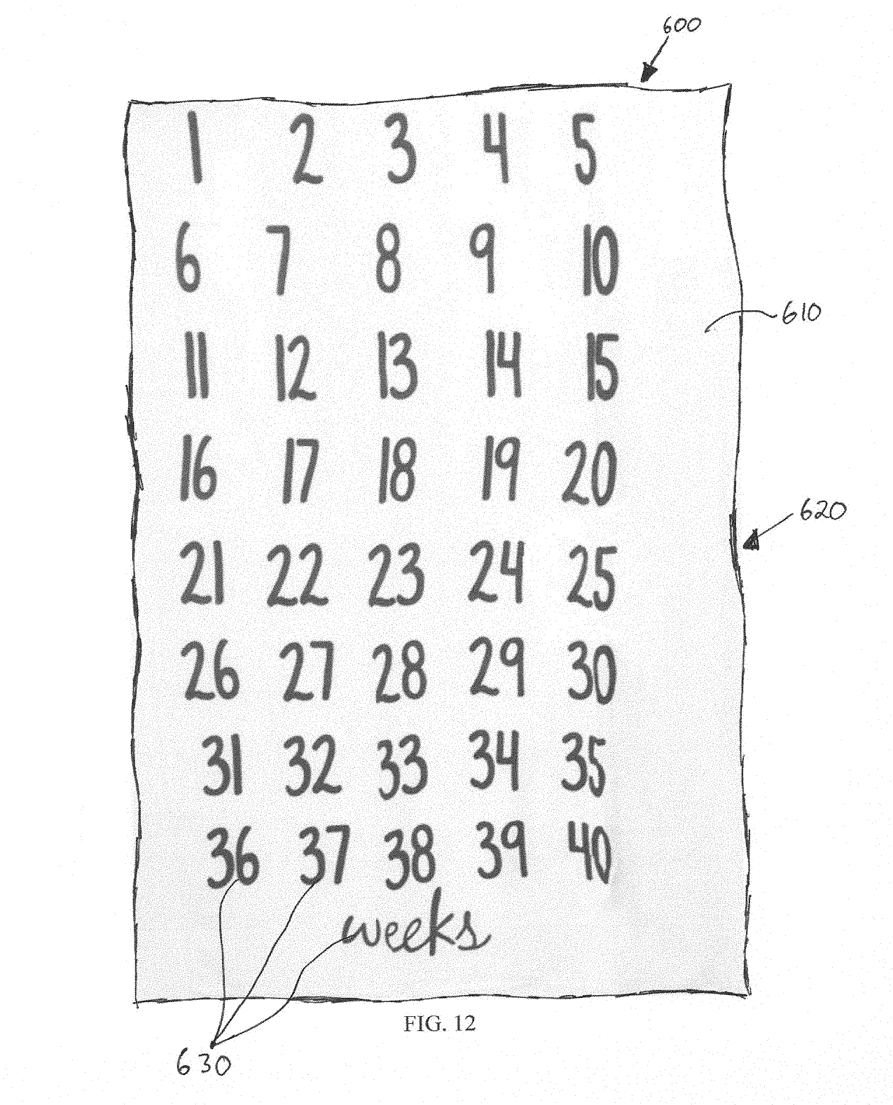

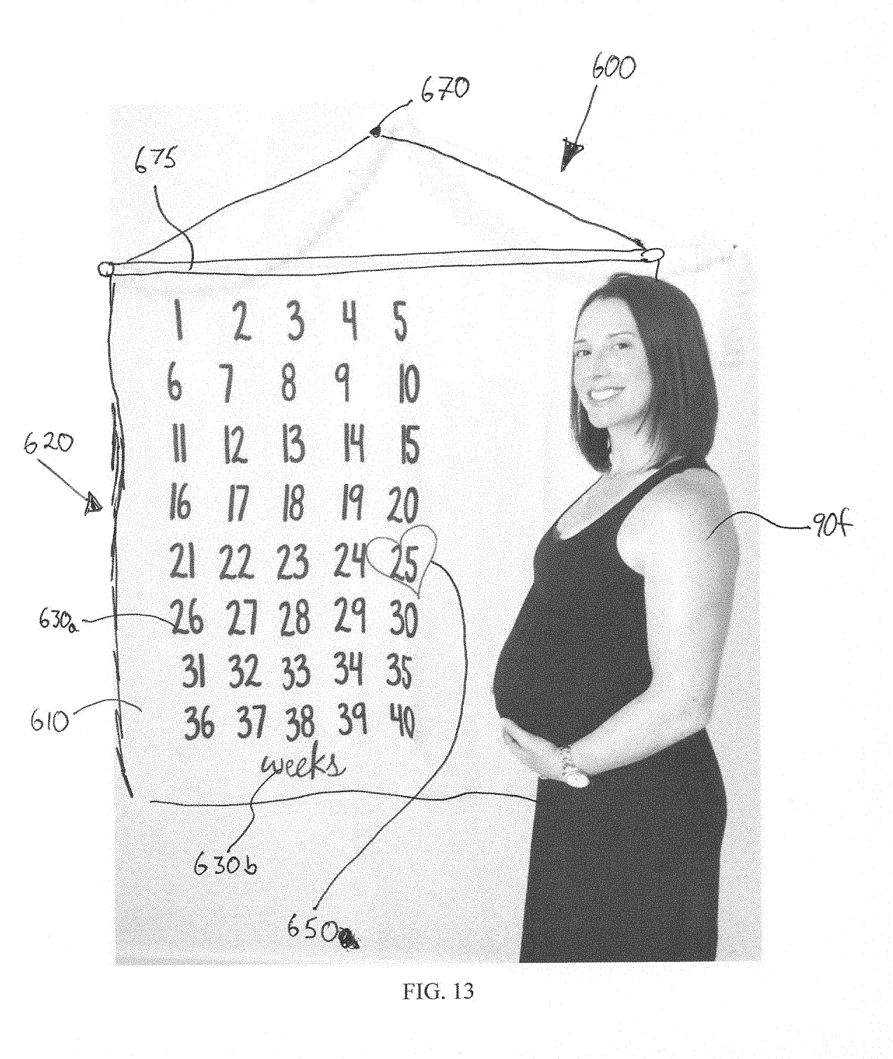

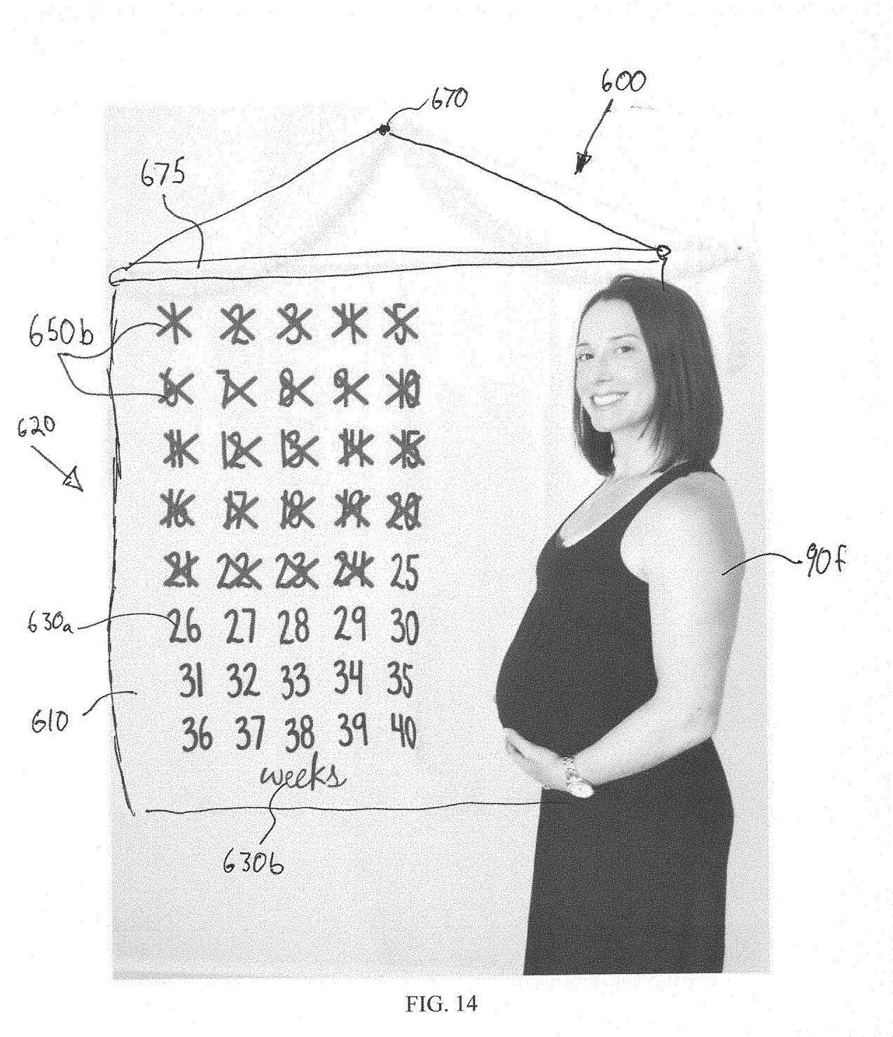

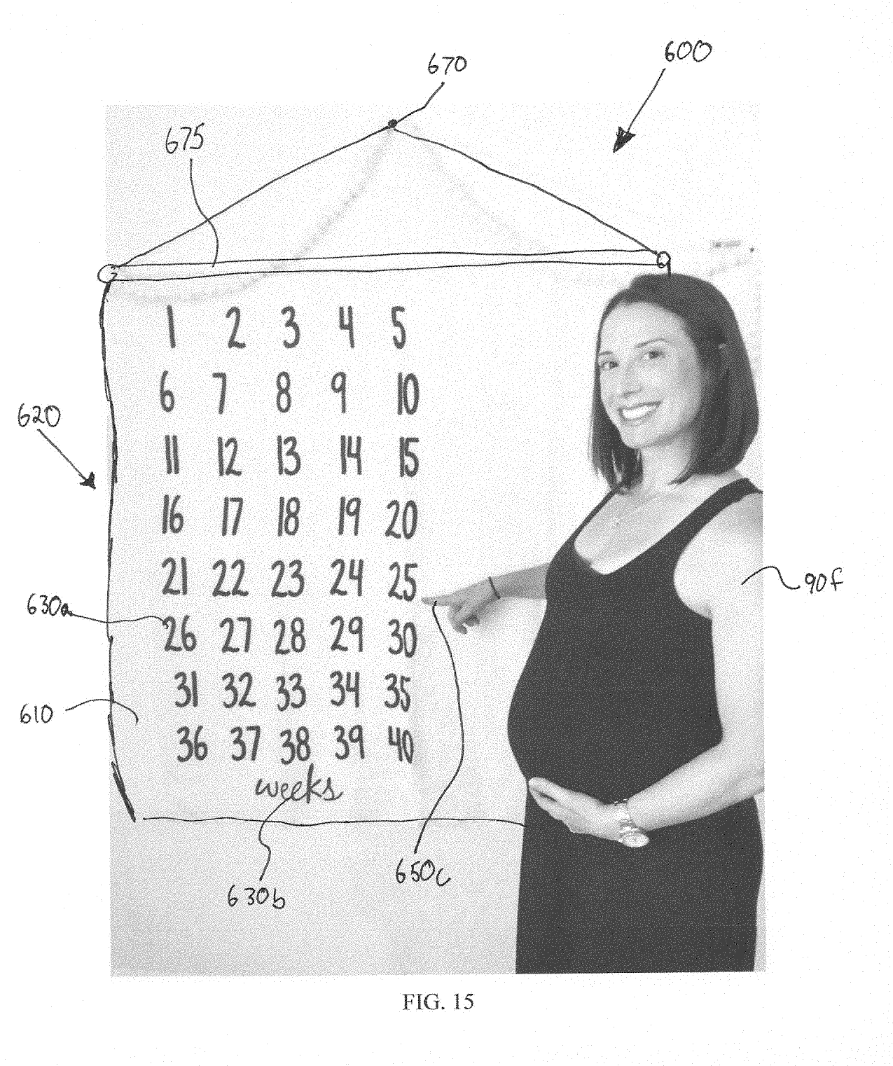

In addition to human growth from infancy to adulthood, human women also experience growth in the torso area during pregnancy. With still continuing reference to the drawings, FIGS. 12-15 depict a human growth tracking apparatus 600 being used in various manners to track the growth of a pregnant human 90f. Here, since a typical pregnancy lasts around 40 weeks, the plurality of indicia 630 are provided with indicia numbers 1-40. Depending on the numbered indicia 630a, a correlation can be drawn between a particular week of pregnancy. The indicia “Weeks” 630b is set forth to clarify the information presented for potential use for tracking purposes. Here also, a support component 670 is used to suspend a rod 675 from which the human growth tracking apparatus embodiment 600 is draped. As shown particularly in FIG. 13, a heart-shaped signifying element 650 is utilized to identify the 25 week as the notable event corresponding to the growing size of the pregnant human 90f. The depiction in FIG. 14 teaches a different approach to identifying significance; all of the indicia 630 prior to week 25 are crossed off 650b, thereby leaving 25 as the week having notable significant in correlation with the growing size of the pregnant human 90f In a different manner, the depiction of FIG. 15 shows the pregnant human 90f pointing to signify that week 25 of her pregnancy correlates with her size as may be recorded via image capture, so that the notable event(s) are signified by directed actions of the human, such as the pregnant human 90f, for whom growth is being tracked. Again, as before, the display face 620 and flexible sheet 610 and indicia 630b are similar to that described with respect to comparable elements associated with human growth tracking apparatus embodiment 100 described and depicted in regard to FIGS. 1-3.

With reference being made to FIG. 1-FIG. 16, methodology corresponding to the tracking human growth in correlation with notable events is described. Once an embodiment of a human growth tracking apparatus 100/200/300/400/500/600 is provided, the growth-size of a human 90 can be tracked in correlation with the occurrence of a notable event. Accordingly, the human 90 may be located near or otherwise positioned, oriented, placed and/or set-upon the display face 120/220/320/420/520/620 of the flexible sheet 110/210/310/410/510/610. The location of the human 90 proximate the display face 120/220/320/420/520/620 of the flexible sheet 110/210/310/410/510/610 may be during or close-in-time to the occurrence of the notable event. For example, if the size of the human 90 is desired to be tracked in conjunction with a particular year, week, day, size, or accomplishment, etc., then the human 90 may be located proximate to the display face 120/220/320/420/520/620 of the flexible sheet 110/210/310/410/510/610 either on the date of the human's 90 notable event, or within a day or so prior to or following that date, so that the size of the growing human 90 will correspond accurately with growth up to that notable date and/or accomplishment. In addition to proximately locating the human 90 next to or on the human growth tracking apparatus embodiment 100/200/300/400/500/600, a user may also signify, circle, point-to, highlight, emphasize, or otherwise recognize specific indicia 150/250/350/450/550/650 that correlatively identifies the particular date and/or notable event being identified as being correspondingly relevant for growth tracking. An imaging device, such as a camera or other device capable of capturing an image, may be provided to capture at least one image of the human 90 located proximate the signified indicia 150/250/350/450/550/650, of the plurality of indicia 130/230/330/430/530/630, thereby identifying the human 90 as being a tracked size on the notable date or day of accomplishment in the captured image. For purposes of documenting or otherwise tracking the size of the human 90 as captured in correlation with the notable moment, the captured image may be at least temporarily stored, such as on film or on a digital hard drive or flash drive, during or close-in-time to the occurrence of the notable event during the period of growth of the human 90.

Although various aspects are illustrated and described herein with reference to specific embodiments, the aspects, in whole and in part, are not intended to be limited to the details shown. For example, the apparatus, system and methods described herein may be utilized to track reduction in human size, as opposed to growth in human size, and may also be used to track any change in human size. Rather, various modifications may be made in the details within the scope and range of equivalents of the claims and without departing from the invention.

What is claimed is:

1. A human growth tracking apparatus comprising:
a flexible tracking sheet, the flexible tracking sheet having at least one display face, wherein the at least one display face covers an area of at least four square feet, the at least four square foot area being defined by at least two perpendicular dimensions, each of the at least two perpendicular dimensions being at least two feet in length; and
a plurality of indicia located on the at least one display face of the flexible tracking sheet, wherein the plurality of indicia corresponds to notable events that occur during a period of growth of a human.

2. The apparatus of claim 1, wherein the plurality of indicia is associated with months.

3. The apparatus of claim 1, wherein the plurality of indicia comprises alphanumeric symbols.

4. The apparatus of claim 1, wherein the plurality of indicia comprises pictorial symbols.

5. The apparatus of claim 1, further comprising support elements for suspending the apparatus vertically.

6. The apparatus of claim 1, wherein the flexible sheet is a swaddle blanket made of bamboo cotton muslin.

7. The apparatus of claim 1, wherein the indicia correspond to weeks of a pregnancy.

8. A system for tracking human growth in correlation with notable events, the system comprising:
an imaging device, the imaging device configured to capture at least one image of a growing human, wherein the imaging device includes a storage medium configured to store the at least one captured image of the growing human, and further wherein the at least one stored image is captured during or close-in-time to the occurrence of a notable event during a period of growth of the human; and
a human growth tracking apparatus, the human growth tracking apparatus including:
a flexible tracking sheet, the flexible tracking sheet having at least one display face, wherein the at least one display face covers an area of at least four square feet, the at least four square foot area being defined by at least two perpendicular dimensions, each of the at least two perpendicular dimensions being at least two feet in length; and
a plurality of indicia located on the at least one display face of the flexible tracking sheet, wherein the plurality of indicia corresponds to notable events that occur during the period of growth of the human.

9. The system of claim 8, wherein the imaging device is a digital camera.

10. The system of claim 8, wherein the plurality of indicia corresponds to several different holidays.

11. The system of claim 10, further comprising accoutrements associated with a particular holiday signified as the noted event.

12. The system of claim 8, wherein the plurality of indicia corresponds to several different milestones reached by a growing infant.

13. The system of claim 8, further comprising extraneous indicia.

14. The system of claim 8, wherein the notable events are signified by directed actions of the human for whom growth is being tracked.

15. The system of claim 8, wherein the plurality of indicia corresponds to each of years, months, weeks and days.

15. A method of tracking human growth in correlation with notable events, the method comprising:
providing a human growth tracking apparatus, the human growth tracking apparatus including;
a flexible tracking sheet, the flexible tracking sheet having at least one display face, wherein the at least one display face covers an area of at least four square feet, the at least four square foot area being defined by at least two perpendicular dimensions, each of the at least two perpendicular dimensions being at least two feet in length; and
a plurality of indicia located on the at least one display face of the flexible tracking sheet, wherein the plurality of indicia corresponds to notable events that occur during a period of growth of a human;
locating a human proximate the plurality of indicia located on the at least one display face of the flexible tracking sheet during or close-in-time to the occurrence of a notable event during the period of growth of the human;
signifying specific indicia that correlatively identifies the particular notable event being identified as correspondingly relevant for tracking growth of the human;
providing an imaging device;
capturing at least one image of the growing human, as the growing human is located proximate the plurality of indicia located on the at least one display face of the flexible tracking sheet, wherein the image is captured during or close-in-time to the occurrence of the notable event during the period of growth of the human; and
storing, at least temporarily, the at least one captured image of the growing human, wherein the captured image is stored during or close-in-time to the occurrence of the notable event during the period of growth of the human.

16. The method of claim 15, wherein the growing human is an infant.

17. The method of claim 15, wherein the growing human is a child.

18. The method of claim 15, wherein the growing human is a pregnant woman.

19. The method of claim 15, wherein the imaging device is a digital camera.

20. The method of claim 15, wherein the human growth tracking apparatus is oriented vertically.
